SEQUENCE LISTING (8 sequences)

SEQ ID NO: 1 - protein, 112 aa, Nocardioides zeae
PSSTEYDADGDGYVDTRESDTDGDGYVDTIETDTDGDGWVDTVATDTDGDGYIDTVATDTDGDGYADVVETDTDGDGYTDEVAYDADGDGYIDTVEADTDGDGYTDTVVHDG

SEQ ID NO: 2 - protein, 137 aa, Nocardioides zeae
MYASNAEPTPPPAPSSTEYDADGDGYVDTRESDTDGDGYVDTIETDTDGDGWVDTVATDTDGDGYIDTVATDTDGDGYADVVETDTDGDGYTDEVAYDADGDGYIDTVEADTDGDGYTDTVVHDGASDSGLESTLDA

SEQ ID NO: 3 - protein, 117 aa, Nocardioides zeae
MGSGPSSTEYDADGDGYVDTRESDTDGDGYVDTIETDTDGDGWVDTVATDTDGDGYIDTVATDTDGDGYADVVETDTDGDGYTDEVAYDADGDGYIDTVEADTDGDGYTDTVVHDGS

SEQ ID NO: 4 - protein, 420 aa, Nocardioides zeae
MGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSGGSGSGSGPSSTEYDADGDGYVDTRESDTDGDGYVDTIETDTDGDGWVDTVATDTDGDGYIDTVATDTDGDGYADVVETDTDGDGYTDEVAYDADGDGYIDTVEADTDGDGYTDTVVHDG

SEQ ID NO: 5 - protein, 176 aa, Nocardioides zeae
MSSGSTNPGVSAWQVNTAYTAGQLVTYNGKTYKCLQPHTSLAGWEPSNVPALWQLQGSSGSSSGPSSTEYDADGDGYVDTRESDTDGDGYVDTIETDTDGDGWVDTVATDTDGDGYIDTVATDTDGDGYADVVETDTDGDGYTDEVAYDADGDGYIDTVEADTDGDGYTDTVVHDG

SEQ ID NO: 6 - protein, 342 aa, Nocardioides zeae
MGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSGGSGSGSGPSSTEYDADGDGYVDTRESDTDGDGYVDTIETDT

SEQ ID NO: 7 - protein, 329 aa, Nocardioides zeae
MGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSGGSGSGSGPSSTEYDADGDGYVDTRESDT

SEQ ID NO: 8 - protein, 25 aa, synthetic construct
SKSGPSSTEYDADGDGYVDTRESDT

(19) United States
(12) Patent Application Publication
(10) Pub. No.: US 2025/0376489 A1
(43) Pub. Date: Dec. 11, 2025

(54) RARE EARTH ELEMENT BINDING PROTEIN

(71) Applicant: Battelle Memorial Institute, Columbus, OH (US)

(72) Inventors: Bradley S. HEATER, Dublin, OH (US); Katarzyna H. KUCHARZYK, Columbus, OH (US); Matt ROWLEY, Columbus, OH (US); Daniel R. MARZOLF, Columbus, OH (US); Jonathan W. PICKING, Hilliard, OH (US); Steven A. HIGGINS, Columbus, OH (US); Scott BANTA, Fairfield, CT (US); Farid KHOURY, New York, NY (US)

(21) Appl. No.: 19/229,829

(22) Filed: Jun. 5, 2025

Related U.S. Application Data
(60) Provisional application No. 63/656,161, filed on Jun. 5, 2024.

(51) Int. Cl. C07K 7/06; G01N 1/40

(52) U.S. Cl. CPC C07K 7/06; G01N 1/4044

(57) ABSTRACT

This invention relates to rare earth element binding protein and methods of recovering a rare earth element (REE) from a sample. The (REE) binding protein comprises the repeating sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N.

### CROSS-REFERENCE TO RELATED APPLICATIONS

The present application claims the benefit of the filing date of U.S. Provisional Application Ser. No. 63/656,161, filed Jun. 5, 2024, the entire teachings of which application is hereby incorporated herein by reference.

### STATEMENT REGARDING FEDERALLY SPONSORED RESEARCH OR DEVELOPMENT

This invention was made with government support under contract number FA8650-22-C-7213 awarded by the Defense Advanced Research Projects Agency. The government has certain rights in the invention.

### SEQUENCE LISTING

The instant application contains a Sequence Listing which has been submitted electronically in XML file format and is hereby incorporated by reference in its entirety. Said XML copy, created on Jun. 5, 2025, is named BAT271US_SL.xml and is 9,321 bytes in size.

### FIELD

This invention relates to rare earth element binding protein and methods of recovering a rare earth element (REE) from a sample.

### BACKGROUND

A challenge in establishing a more diversified REE supply chain is the difficulty of achieving cost-effective and environmentally sustainable REE extraction and separation from ore deposits and REE-containing waste. The current industrial REE production processes generate radioactive wastes, high volumes of acidic effluents, and organic solvents, resulting in a severe environmental burden. To alleviate supply vulnerability and diversify the global REE production chain, new processing technologies, specifically based in biological advancements enabling green REE extraction from alternative REE resources, are desired.

Proteins offer highly specific environments for metal-biological interactions to occur, so there is significant interest in their use for eco-friendly REE separation and purification. One such protein of interest is Lanmodulin (LanM), which reportedly demonstrates significantly higher binding affinity for REEs compared to calcium and other contaminant metals. However, LanM only binds 1-2 REEs when immobilized and exhibits relatively poor selectivity for individual REEs. See, e.g., WO2022/266120 entitled Compositions Comprising Proteins And Methods OF Use Thereof For Rare Earth Element Separation.

### SUMMARY

A rare earth element (REE) binding protein comprising the repeating sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N.

A rare earth element (REE) binding protein wherein the REE-binding protein comprises a sequence with at least 75% identity to SEQ ID NO. 1.

A method of recovering a rare earth element (REE) from a sample comprising:
a. introducing a REE-binding protein to the sample wherein the REE-binding protein comprises the repeating sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N;
b. recovering the REE from the sample.

A method of recovering a rare earth element (REE) from a sample comprising:
a. introducing a REE-binding protein to the sample wherein the REE-binding protein comprises a sequence with at least 75% identity to SEQ ID NO. 1; and
b. recovering the REE from the sample.

A method of recovering a rare earth element (REE) from a sample comprising:
a. providing REE-binding protein immobilized on a solid matrix where the REE binding protein comprises the repeating sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N;
b. introducing a sample containing one or more REEs onto said REE-binding protein immobilized on said solid matrix;
c. loading said one or more REEs onto said REE-binding protein immobilized on said solid matrix;
d. unloading said one or more REEs from said REE-binding protein immobilized on said solid matrix.

A method of recovering a rare earth element (REE) from a sample comprising:
a. providing REE-binding protein immobilized on a solid matrix where the REE-binding protein comprises a sequence with at least 75% identity to SEQ ID NO. 1
b. introducing a sample containing one or more REEs onto said REE-binding protein immobilized on said solid matrix;
c. loading said one or more REEs onto said REE-binding protein immobilized on said solid matrix; and
d. unloading said one or more REEs from said REE-binding protein immobilized on said solid matrix.

### DRAWINGS

FIG. 1 provides an AlaphaFold2 model of the REE-binding protein comprising SEQ ID NO. 1 (HEW5).

FIG. 2 illustrates the binding performance of the REE-binding protein comprising SEQ ID NO. 1 (HEW5).

FIG. 3 illustrates the separation of the indicated REEs using an immobilized REE-binding protein having SEQ ID NO. 1.

FIG. 4 depicts separation of pre-filtered Ce-removed simulated leachate using immobilized SEQ ID NO. 1 via the Halotag system (SEQ ID NO. 4) using a pH gradient. The figure shows ICP-MS validated data for a single cycle. Each data point was obtained via ICP-MS analysis.

FIG. 5 illustrates recyclability of SEQ ID NO. 1 by binding and eluting Nd.

FIG. 6 describes binding capacity of SEQ ID NO. 1 column determined via saturation to be nearly 17 μ moles of REE binding capacity. The shaded area represents the eluted fractions used in the calculation.

FIG. 7a demonstrates that the individual repeating domains (i.e. X1-X9) are functional albeit have less REE loading capacity.

FIG. 7b shows that the selectivity of the individual domains does not change much compared to full length HEW5.

### DETAILED DESCRIPTION OF PREFERRED EMBODIMENTS

REEs comprise a group of metals including lanthanides, yttrium (Y), and scandium (Sc). The lanthanides (or lanthanoids) are elements with atomic numbers 57 through 71 (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Flo), erbium (Er), thulium (Tm), ytterbium (Yb), and lutetium (Lu), respectively).

The present invention provides a REE-binding protein, which may be utilized for example, in the recovery of REEs from a sample. The preferred REE herein has a molecular weight of 11.7 kDa and has eight (8) metal binding sites. The protein preferably binds REEs in the range of 10 μM to 50 μM. In addition, the REE-binding protein herein preferably provides a selectivity toward light rare earth elements (i.e., La, Ce, Pr, Nd, Pm, Sm, Eu, Gd) as compared to heavy rare earth elements (i.e., Tb, Dy, Ho, Er, Tm, Yb, Lu). More preferably, the preference amounts to a 2-4 fold selectivity preference toward light REE compared to heavy REE. The REE-binding protein herein also preferably allows for REE separation without the use of chelators.

The REE binding protein herein (HEW5) may first be described as continuous sequence of at least nine (9) amino acids with the sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N. More preferably, the aforementioned sequence is contemplated to repeat 2, 4, 6, 8, 10 or 12 times, and such repeating sequence is preferably separated by at least four (4) amino acids. HEW5 contains 8 REE binding sites—the capacity was empirically determined to be 17μ mol by overloading the HEW5 column with La, washing unbound REE and then eluting at pH 3.0. Notably, this capacity matches the theoretical molar capacity based on the 8 binding sites and amount of HEW5 loaded per mL beads in the column.

We have also demonstrated that the individual domain within HEW5 (i.e. X₁-X₉) is functional. Halo-HEW3.2 contains two metal binding sites, Halo-HEW1.6 contains a single metal binding site. Both can be immobilized and are capable of binding REEs, though the total amount of REEs decreases with decreasing number of binding sites. However, the proteins still show selectivity similar to full length HEW5, with slight differences. Additionally, a short peptide was synthesized that contains the same X₁-X₉ motif, immobilized via an incorporated lysine residue, and characterized. It showed similar binding capacity as Halo-HEW1.6.

The REE-binding protein herein is preferably truncated from the 137 amino acid full-length protein from Nocardioides zeae (SEQ ID NO. 2). The REE-binding protein therefore preferably has the domain sequence selected from SEQ ID NO. 1 and can be expressed in E. coli using coding SEQ ID NO. 3.:

```
SEQ ID NO 1 (TRUNCATED HEW5)
LENGTH: 112
TYPE: PRT
ORGANISM: Nocardioides zeae
SEQUENCE: 1
Pro Ser Ser Thr Glu Tyr Asp Ala Asp Gly Asp Gly Tyr Val Asp
1 5 10 15
Thr Arg Glu Ser Asp Thr Asp Gly Asp Gly Tyr Val Asp Thr Ile
                                20 25 30
Glu Thr Asp Thr Asp Gly Asp Gly Trp Val Asp Thr Val Ala Thr
                                35 40 45
Asp Thr Asp Gly Asp Gly Tyr Ile Asp Thr Val Ala Thr Asp Thr
                                50 55 60
Asp Gly Asp Gly Tyr Ala Asp Val Val Glu Thr Asp Thr Asp Gly
                                65 70 75
Asp Gly Tyr Thr Asp Glu Val Ala Tyr Asp Ala Asp Gly Asp Gly
                                80 85 90
Tyr Ile Asp Thr Val Glu Ala Asp Thr Asp Gly Asp Gly Tyr Thr
                                95 100 105
Asp Thr Val Val His Asp Gly
                                110
SEQ ID NO 2 (WT HEW5)
LENGTH: 137
TYPE: PRT
ORGANISM: Nocardioides zeae
SEQUENCE: 1
Met Tyr Ala Ser Asn Ala Glu Pro Thr Pro Pro Pro Ala Pro
1 5 10 15
Ser Thr Glu Tyr Asp Ala Asp Gly Asp Gly Tyr Val Asp Thr Arg
                                20 25 30
Glu Ser Asp Thr Asp Gly Asp Gly Tyr Val Asp Thr Ile Glu Thr
                                35 40 45
Asp Thr Asp Gly Asp Gly Trp Val Asp Thr Val Ala Thr Asp Thr
                                50 55 60
Asp Gly Asp Gly Tyr Ile Asp Thr Val Ala Thr Asp Thr Asp Gly
                                65 70 75
Asp Gly Tyr Ala Asp Val Val Glu Thr Asp Thr Asp Gly Asp Gly
                                80 85 90
Tyr Thr Asp Glu Val Ala Tyr Asp Ala Asp Gly Asp Gly Tyr Ile
                                95 100 105
Asp Thr Val Glu Ala Asp Thr Asp Gly Asp Gly Tyr Thr Asp Thr
                                110 115 120
Val Val His Asp Gly Ala Ser Asp Ser Gly Leu Glu Ser Thr Leu
                                125 130 135
Asp Ala
SEQ ID NO 3 (E. coli HEW5)
LENGTH: 117
TYPE: PRT
ORGANISM: Nocardioides zeae
SEQUENCE: 1
Met Gly Ser Gly Pro Ser Ser Thr Glu Tyr Asp Ala Asp Gly Asp
1 5 10 15
Gly Tyr Val Asp Thr Arg Glu Ser Asp Thr Asp Gly Asp Gly Tyr
                                20 25 30
Val Asp Thr Ile Glu Thr Asp Thr Asp Gly Asp Gly Trp Val Asp
                                35 40 45
Thr Val Ala Thr Asp Thr Asp Gly Asp Gly Tyr Ile Asp Thr Val
                                50 55 60
Ala Thr Asp Thr Asp Gly Asp Gly Tyr Ala Asp Val Val Glu Thr
                                65 70 75
Asp Thr Asp Gly Asp Gly Tyr Thr Asp Glu Val Ala Tyr Asp Ala
                                80 85 90
Asp Gly Asp Gly Tyr Ile Asp Thr Val Glu Ala Asp Thr Asp Gly
                                95 100 105
Asp Gly Tyr Thr Asp Thr Val Val His Asp Gly Ser
                                110 115
SEQ ID NO 4 (HALO HEW5)
LENGTH: 420
TYPE: PRT
ORGANISM: Nocardioides zeae
SEQUENCE: 1
Met Gly Ser Glu Ile Gly Thr Gly Phe Pro Phe Asp Pro His Tyr
1 5 10 15
Val Glu Val Leu Gly Glu Arg Met His Tyr Val Asp Val Gly Pro
                                20 25 30
Arg Asp Gly Thr Pro Val Leu Phe Leu His Gly Asn Pro Thr Ser
                                35 40 45
Ser Tyr Val Trp Arg Asn Ile Ile Pro His Val Ala Pro Thr His
                                50 55 60
Arg Cys Ile Ala Pro Asp Leu Ile Gly Met Gly Lys Ser Asp Lys
                                65 70 75
Pro Asp Leu Gly Tyr Phe Phe Asp Asp His Val Arg Phe Met Asp
                                80 85 90
Ala Phe Ile Glu Ala Leu Gly Leu Glu Glu Val Val Leu Val Ile
                                95 100 105
His Asp Trp Gly Ser Ala Leu Gly Phe His Trp Ala Lys Arg Asn
                                110 115 120
Pro Glu Arg Val Lys Gly Ile Ala Phe Met Glu Phe Ile Arg Pro
                                125 130 135
Ile Pro Thr Trp Asp Glu Trp Pro Glu Phe Ala Arg Glu Thr Phe
                                140 145 150
Gln Ala Phe Arg Thr Thr Asp Val Gly Arg Lys Leu Ile Ile Asp
                                155 160 165
Gln Asn Val Phe Ile Glu Gly Thr Leu Pro Met Gly Val Val Arg
                                170 175 180
Pro Leu Thr Glu Val Glu Met Asp His Tyr Arg Glu Pro Phe Leu
                                185 190 195
Asn Pro Val Asp Arg Glu Pro Leu Trp Arg Phe Pro Asn Glu Leu
                                200 205 210
Pro Ile Ala Gly Glu Pro Ala Asn Ile Val Ala Leu Val Glu Glu
                                215 220 225
Tyr Met Asp Trp Leu His Gln Ser Pro Val Pro Lys Leu Leu Phe
                                230 235 240
Trp Gly Thr Pro Gly Val Leu Ile Pro Pro Ala Glu Ala Ala Arg
                                245 250 255
Leu Ala Lys Ser Leu Pro Asn Cys Lys Ala Val Asp Ile Gly Pro
                                260 265 270
Gly Leu Asn Leu Leu Gln Glu Asp Asn Pro Asp Leu Ile Gly Ser
                                275 280 285
Glu Ile Ala Arg Trp Leu Ser Thr Leu Glu Ile Ser Gly Gly Ser
                                290 295 300
Gly Gly Ser Gly Ser Gly Ser Gly Pro Ser Ser Thr Glu Tyr Asp
                                305 310 315
Ala Asp Gly Asp Gly Tyr Val Asp Thr Arg Glu Ser Asp Thr Asp
                                320 325 330
Gly Asp Gly Tyr Val Asp Thr Ile Glu Thr Asp Thr Asp Gly Asp
                                335 340 345
Gly Trp Val Asp Thr Val Ala Thr Asp Thr Asp Gly Asp Gly Tyr
                                350 355 360
Ile Asp Thr Val Ala Thr Asp Thr Asp Gly Asp Gly Tyr Ala Asp
                                365 370 375
Val Val Glu Thr Asp Thr Asp Gly Asp Gly Tyr Thr Asp Glu Val
                                380 385 390
Ala Tyr Asp Ala Asp Gly Asp Gly Tyr Ile Asp Thr Val Glu Ala
                                395 400 405
Asp Thr Asp Gly Asp Gly Tyr Thr Asp Thr Val Val His Asp Gly
                                410 415 420
SEQ ID NO 5 (CBD HEW5)
LENGTH: 176
TYPE: PRT
ORGANISM: Nocardioides zeae
SEQUENCE: 1
Met Ser Ser Gly Ser Thr Asn Pro Gly Val Ser Ala Trp Gln Val
1 5 10 15
Asn Thr Ala Tyr Thr Ala Gly Gln Leu Val Thr Tyr Asn Gly Lys
                                20 25 30
Thr Tyr Lys Cys Leu Gln Pro His Thr Ser Leu Ala Gly Trp Glu
                                35 40 45
Pro Ser Asn Val Pro Ala Leu Trp Gln Leu Gln Gly Ser Ser Gly
                                50 55 60
Ser Ser Ser Gly Pro Ser Ser Thr Glu Tyr Asp Ala Asp Gly Asp
                                65 70 75
Gly Tyr Val Asp Thr Arg Glu Ser Asp Thr Asp Gly Asp Gly Tyr
                                80 85 90
Val Asp Thr Ile Glu Thr Asp Thr Asp Gly Asp Gly Trp Val Asp
                                95 100 105
Thr Val Ala Thr Asp Thr Asp Gly Asp Gly Tyr Ile Asp Thr Val
                                110 115 120
Ala Thr Asp Thr Asp Gly Asp Gly Tyr Ala Asp Val Val Glu Thr
                                125 130 135
Asp Thr Asp Gly Asp Gly Tyr Thr Asp Glu Val Ala Tyr Asp Ala
                                140 145 150
Asp Gly Asp Gly Tyr Ile Asp Thr Val Glu Ala Asp Thr Asp Gly
                                155 160 165
Asp Gly Tyr Thr Asp Thr Val Val His Asp Gly
                                170
SEQ ID NO 6 (HALO HEW3.2)
LENGTH: 342
TYPE: PRT
ORGANISM: Nocardioides zeae
SEQUENCE: 1
Met Gly Ser Glu Ile Gly Thr Gly Phe Pro Phe Asp Pro His Tyr
1 5 10 15
Val Glu Val Leu Gly Glu Arg Met His Tyr Val Asp Val Gly Pro
                                20 25 30
Arg Asp Gly Thr Pro Val Leu Phe Leu His Gly Asn Pro Thr Ser
                                35 40 45
Ser Tyr Val Trp Arg Asn Ile Ile Pro His Val Ala Pro Thr His
                                50 55 60
Arg Cys Ile Ala Pro Asp Leu Ile Gly Met Gly Lys Ser Asp Lys
                                65 70 75
Pro Asp Leu Gly Tyr Phe Phe Asp Asp His Val Arg Phe Met Asp
                                80 85 90
Ala Phe Ile Glu Ala Leu Gly Leu Glu Glu Val Val Leu Val Ile
                                95 100 105
His Asp Trp Gly Ser Ala Leu Gly Phe His Trp Ala Lys Arg Asn
                                110 115 120
Pro Glu Arg Val Lys Gly Ile Ala Phe Met Glu Phe Ile Arg Pro
                                125 130 135
Ile Pro Thr Trp Asp Glu Trp Pro Glu Phe Ala Arg Glu Thr Phe
                                140 145 150
Gln Ala Phe Arg Thr Thr Asp Val Gly Arg Lys Leu Ile Ile Asp
                                155 160 165
Gln Asn Val Phe Ile Glu Gly Thr Leu Pro Met Gly Val Val Arg
                                170 175 180
Pro Leu Thr Glu Val Glu Met Asp His Tyr Arg Glu Pro Phe Leu
                                185 190 195
Asn Pro Val Asp Arg Glu Pro Leu Trp Arg Phe Pro Asn Glu Leu
                                200 205 210
Pro Ile Ala Gly Glu Pro Ala Asn Ile Val Ala Leu Val Glu Glu
                                215 220 225
Tyr Met Asp Trp Leu His Gln Ser Pro Val Pro Lys Leu Leu Phe
                                230 235 240
Trp Gly Thr Pro Gly Val Leu Ile Pro Pro Ala Glu Ala Ala Arg
                                245 250 255
Leu Ala Lys Ser Leu Pro Asn Cys Lys Ala Val Asp Ile Gly Pro
                                260 265 270
Gly Leu Asn Leu Leu Gln Glu Asp Asn Pro Asp Leu Ile Gly Ser
                                275 280 285
Glu Ile Ala Arg Trp Leu Ser Thr Leu Glu Ile Ser Gly Gly Ser
                                290 295 300
Gly Gly Ser Gly Ser Gly Ser Gly Pro Ser Ser Thr Glu Tyr Asp
                                305 310 315
Ala Asp Gly Asp Gly Tyr Val Asp Thr Arg Glu Ser Asp Thr Asp
                                320 325 330
Gly Asp Gly Tyr Val Asp Thr Ile Glu Thr Asp Thr
                                335 340
SEQ ID NO 7 (HALO HEW1.6)
LENGTH: 329
TYPE: PRT
ORGANISM: Nocardioides zeae
SEQUENCE: 1
Met Gly Ser Glu Ile Gly Thr Gly Phe Pro Phe Asp Pro His Tyr
1 5 10 15
Val Glu Val Leu Gly Glu Arg Met His Tyr Val Asp Val Gly Pro
                                20 25 30
Arg Asp Gly Thr Pro Val Leu Phe Leu His Gly Asn Pro Thr Ser
                                35 40 45
Ser Tyr Val Trp Arg Asn Ile Ile Pro His Val Ala Pro Thr His
                                50 55 60
Arg Cys Ile Ala Pro Asp Leu Ile Gly Met Gly Lys Ser Asp Lys
                                65 70 75
Pro Asp Leu Gly Tyr Phe Phe Asp Asp His Val Arg Phe Met Asp
                                80 85 90
Ala Phe Ile Glu Ala Leu Gly Leu Glu Glu Val Val Leu Val Ile
                                95 100 105
His Asp Trp Gly Ser Ala Leu Gly Phe His Trp Ala Lys Arg Asn
                                110 115 120
Pro Glu Arg Val Lys Gly Ile Ala Phe Met Glu Phe Ile Arg Pro
                                125 130 135
Ile Pro Thr Trp Asp Glu Trp Pro Glu Phe Ala Arg Glu Thr Phe
                                140 145 150
Gln Ala Phe Arg Thr Thr Asp Val Gly Arg Lys Leu Ile Ile Asp
                                155 160 165
Gln Asn Val Phe Ile Glu Gly Thr Leu Pro Met Gly Val Val Arg
                                170 175 180
Pro Leu Thr Glu Val Glu Met Asp His Tyr Arg Glu Pro Phe Leu
                                185 190 195
Asn Pro Val Asp Arg Glu Pro Leu Trp Arg Phe Pro Asn Glu Leu
                                200 205 210
Pro Ile Ala Gly Glu Pro Ala Asn Ile Val Ala Leu Val Glu Glu
                                215 220 225
Tyr Met Asp Trp Leu His Gln Ser Pro Val Pro Lys Leu Leu Phe
                                230 235 240
Trp Gly Thr Pro Gly Val Leu Ile Pro Pro Ala Glu Ala Ala Arg
                                245 250 255
Leu Ala Lys Ser Leu Pro Asn Cys Lys Ala Val Asp Ile Gly Pro
                                260 265 270
Gly Leu Asn Leu Leu Gln Glu Asp Asn Pro Asp Leu Ile Gly Ser
                                275 280 285
Glu Ile Ala Arg Trp Leu Ser Thr Leu Glu Ile Ser Gly Gly Ser
                                290 295 300
Gly Gly Ser Gly Ser Gly Ser Gly Pro Ser Ser Thr Glu Tyr Asp
                                305 310 315
Ala Asp Gly Asp Gly Tyr Val Asp Thr Arg Glu Ser Asp Thr
                                320 325
SEQ ID NO 8 (HALO HEW1.6 Peptide)
LENGTH: 25
TYPE: PRT
ORGANISM: NA
SEQUENCE: 1
Ser Lys Ser Gly Pro Ser Ser Thr Glu Tyr Asp Ala Asp Gly Asp Gly Tyr Val Asp
1 5 10 15
Thr Arg Glu Ser Asp Thr
  20 25
```

In certain embodiments, the REE-binding protein comprises, consists of, or consists essentially of the amino acid sequence set forth in SEQ ID NO. 1, or a sequence with at least 75%, 80%, 85%, 90%, 91%, 92%, 93%, 94%, 95%, 96%, 97%, 98%, or 99% identity to SEQ ID NO. 1.

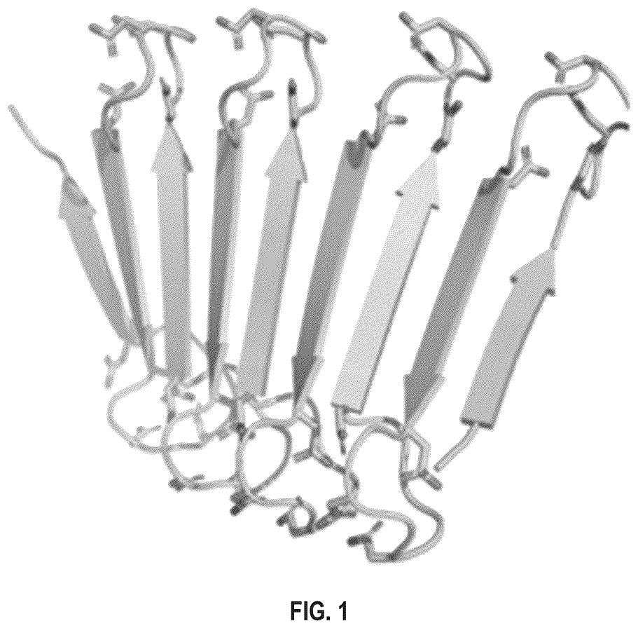

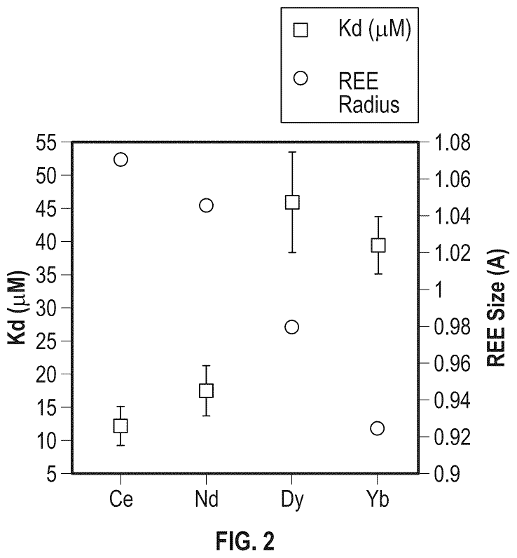

FIG. 1 provides an AlphaFold2 model of the REE-binding protein herein comprising SEQ ID NO. 1. FIG. 2 illustrates the binding performance of the REE-binding protein comprising SEQ ID. NO. 1 illustrating the selectivity towards light REEs as compared to heavy REEs.

Immobilization of the REE-binding protein herein on a solid matrix was confirmed to facilitate REE separation in a continuous flow process. A tagged REE-binding protein having SEQ ID. NO. 1 can be immobilized on a solid matrix such as a packed bead matrix via a non-covalent linkage (Chitin-binding domain via SEQ ID NO. 5 interaction with chitin) or a covalent linkage (performed through Halotag-based immobilization via SEQ ID NO. 4 or directly via lysine in the chitin binding domain of SEQ ID NO. 5 to a bead bearing an oxirane functional group) and a buffer (e.g., pH 5.5) is continuously flowed through the column. Over time, REEs are released from the column using a lower pH buffer (e.g., pH 3.0).

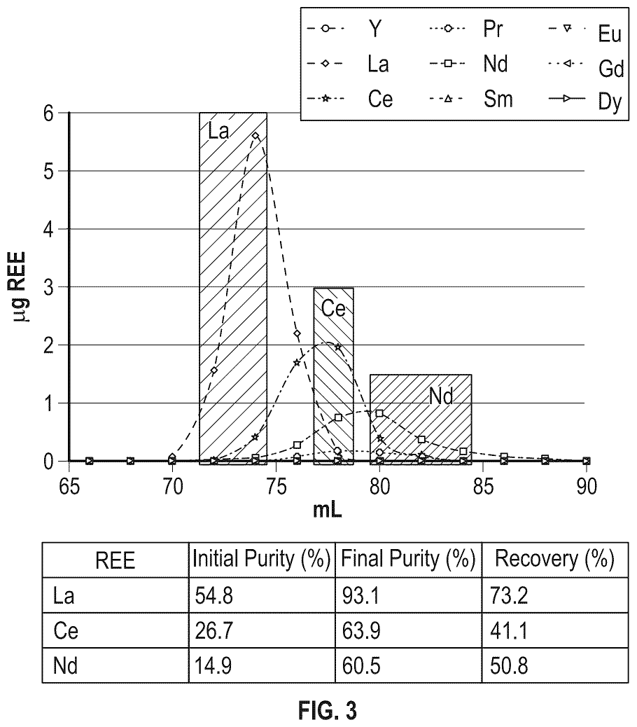

FIG. 3 illustrates the separation of the indicated REEs using the immobilized REE-binding protein having SEQ ID. NO. 1 in a continuous flow system. As can be observed, in a single run three (3) different light REEs could be partially separated, with each nearing 50% recovery and a significant increase in purity compared to the leachate.

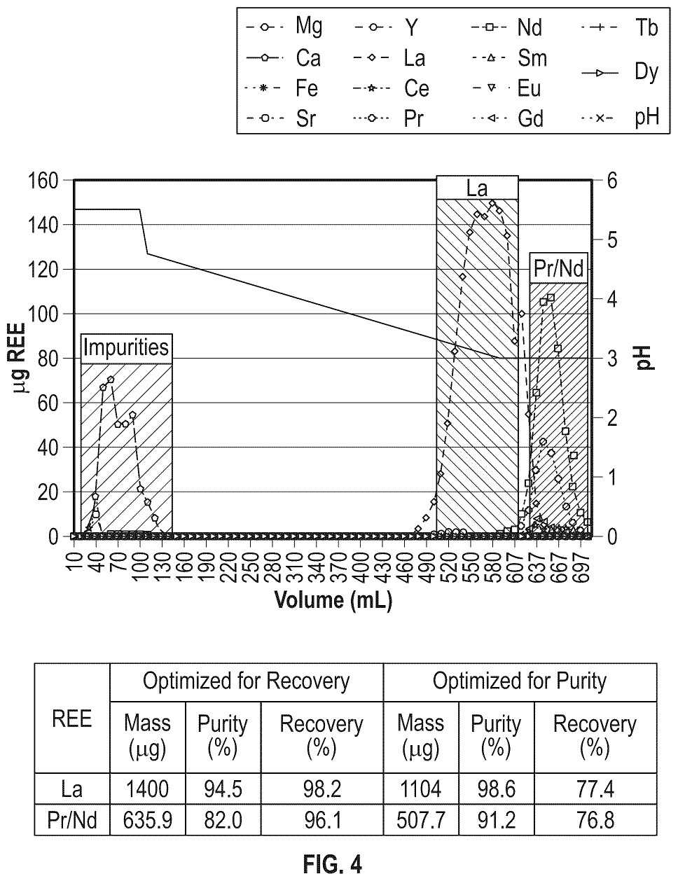

FIG. 4 illustrates the separation of La from Pr and Nd using a mock industrial feedstock, reaching >98% purity of La in a single column run, which demonstrates SEQ ID NO. 1′s industrial utility to separate REEs in commercial feedstocks. Importantly, all impurities (Fe, Sr, Ca, and Mg) were removed early in the pH 5.5 wash. Lastly, stability of the REE-binding protein is important for industrial applications since recovery and reuse of the immobilized protein would reduce process costs.

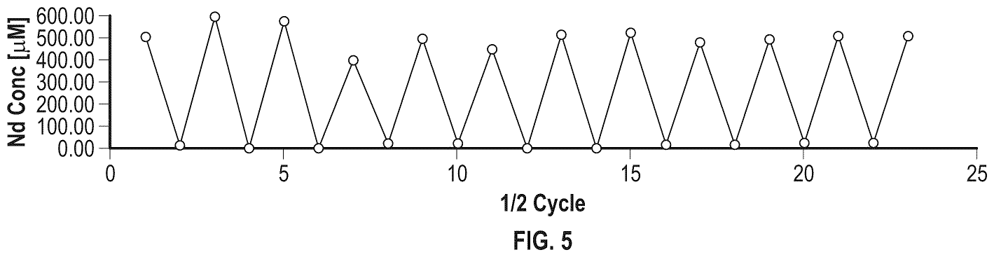

FIG. 5 demonstrates that SEQ ID. No. 1 is capable of ≥10 bind and release cycles of Nd without loss in binding efficiency, indicating that this protein is relatively stable and can be reused for multiple cycles.

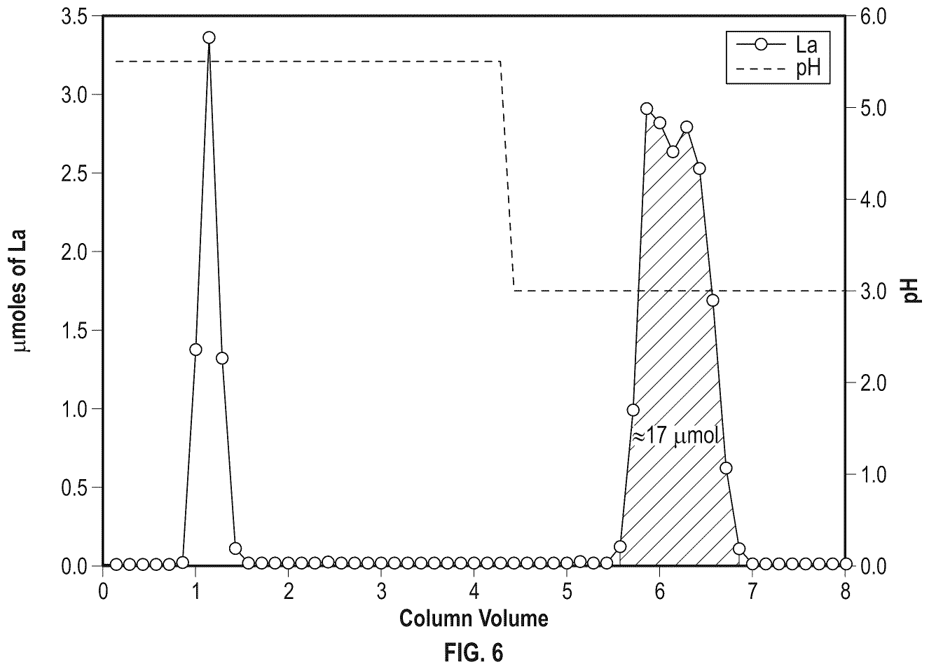

FIG. 6 describes binding capacity of HEW5 column determined via saturation to be nearly 17μ moles of REE binding capacity. The shaded area represents the eluted fractions used in the calculation.

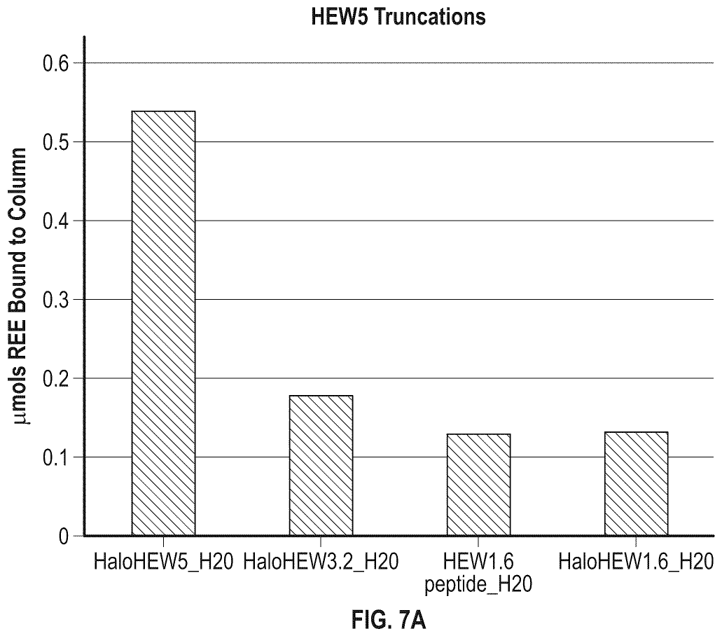

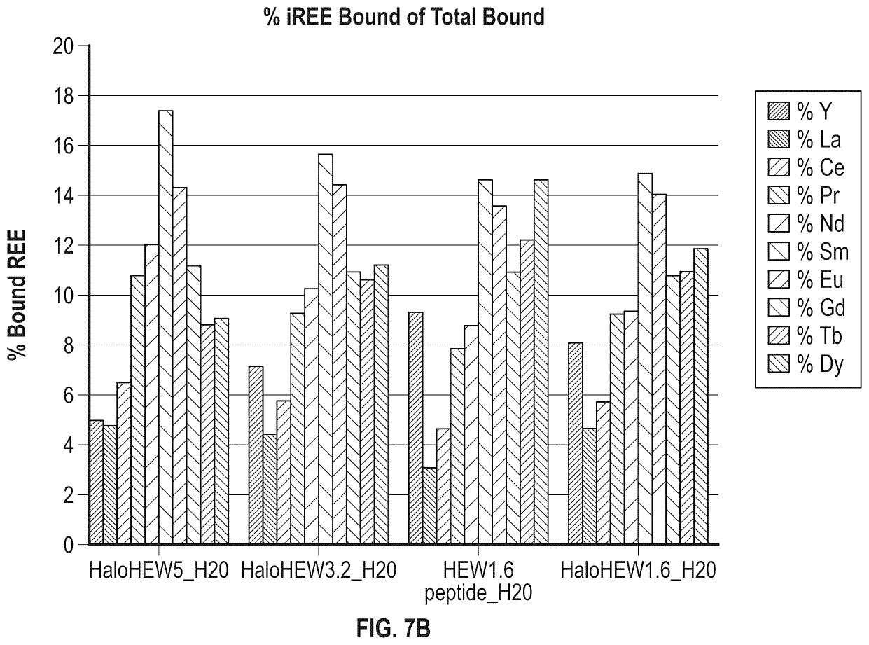

FIG. 7a shows the binding capacity comparison of Halo-HEW5 (8 binding sites), Halo-HEW3.2 (2 binding sites) and a HEW1.6 (1 binding site) in the form of a recombinant fusion (Halo-HEW1.6) and solid-phase synthesized peptide (HEW1.6 peptide). FIG. 7b demonstrates that the truncated proteins display selectivity for different REEs with similar trends as HEW5, albeit some relatively slight differences were observed.

1. A rare earth element (REE) binding protein comprising the repeating sequence X₁X₂X₃X₄X₅X₆X₇X₈X₉ wherein X denotes any amino acid, and X1 is D or E, X2 is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N.

2. A rare earth element (REE) binding protein wherein the REE binding protein comprises a sequence with at least 75% identity to SEQ ID. NO. 1.

3. The REE binding protein of claim 1 wherein the REE binding protein is immobilized on a solid matrix.

4. The REE binding protein of claim 2 wherein the REE binding protein is immobilized on a solid matrix.

5. The REE binding protein of claim 1 wherein said sequence repeats 2, 4, 6, 8, 10 or 12 times.

6. The REE binding protecting of claim 1 wherein the sequence is separated by at least four (4) amino acids.

7. The REE binding protein of claim 1 wherein the REE binding protein is immobilized on said solid matrix via a non-covalent linkage.

8. The REE binding protein of claim 2 wherein the REE binding protein is immobilized on said solid matrix via a non-covalent linkage.

9. The REE binding protein of claim 1 wherein the REE binding protein is immobilized on said solid matrix via a covalent linkage.

10. The REE binding protein of claim 2 wherein the REE binding protein is immobilized on said solid matrix via a covalent linkage.

11. A method of recovering a rare earth element (REE) from a sample comprising:
a. introducing a REE-binding protein to the sample wherein the REE-binding protein comprises a repeating sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N;
b. recovering the REE from the sample.

12. The method of claim 11 further comprising purifying a REE from the sample.

13. The method of claim 11 wherein the REE-binding protein is produced in a host cell and truncated.

14. The method of claim 11 wherein the REE-binding protein indicates a difference in selectivity toward light REEs versus heavy REEs.

15. The method of claim 11 wherein said repeating sequence repeats 2, 4, 6, 8, 10 or 12 times.

16. The method of claim 11 wherein the repeating sequence is separated by at least four (4) amino acids.

17. The method of claim 14 wherein the light REE comprises La, Ce, Pr, Nd, Pm, Sm, Eu, or Gd.

18. The method of claim 14 wherein the heavy rare earth element comprises Tb, Dy, Ho, Er, Tm, Yb, or Lu.

19. A method of recovering a rare earth element (REE) from a sample comprising:
a. introducing a REE-binding protein to the sample wherein the REE-binding protein comprises a sequence with at least 75% identity to SEQ ID NO. 1; and
b. recovering the REE from the sample.

20. The method of claim 19 wherein the REE-binding protein indicates a difference in selectivity toward light REEs versus heavy REEs.

21. The method of claim 20 wherein the light REE comprises La, Ce, Pr, Nd, Pm, Sm, Eu, or Gd.

22. The method of claim 20 wherein the heavy REE comprises Tb, Dy, Ho, Er, Tm, Yb, or Lu.

23. A method of recovering a rare earth element (REE) from a sample comprising:
a. providing REE-binding protein immobilized on a solid matrix where the REE binding protein comprises the repeating sequence X₁X₂X₃X₄X₅X₆X₇X₈X₉ wherein X denotes any amino acid, and X₁ is D or E, X₂ is A, T, or S, X₃ is D, E or N, X₄ is G, A, or F, X₅ is D, X₆ is G, S, or D, X₇ is Y, L, V, F, I, E or W, X₈ is A, V, I, L, F or T, X₉ is D, E, or N;
b. introducing a sample containing one or more REEs onto said REE-binding protein immobilized on said solid matrix;
c. loading said one or more REEs onto said REE-binding protein immobilized on said solid matrix;
d. unloading said one or more REEs from said REE-binding protein immobilized on said solid matrix.

24. The method of claim 23 wherein said unloading of said one or more REEs from said REE-binding protein comprises adjusting pH.

25. A method of recovering a rare earth element (REE) from a sample comprising:
a. providing REE-binding protein immobilized on a solid matrix where the REE-binding protein comprises a sequence with at least 75% identity to SEQ ID NO. 1
b. introducing a sample containing one or more REEs onto said REE-binding protein immobilized on said solid matrix;
c. loading said one or more REEs onto said REE-binding protein immobilized on said solid matrix; and
d. unloading said one or more REEs from said REE-binding protein immobilized on said solid matrix.

26. The method of claim 25 wherein said unloading of said one or more REEs from said REE-binding protein comprises adjusting pH.
